(19) United States
(12) Patent Application Publication
(10) Pub. No.: US 2008/0140138 A1
(43) Pub. Date: Jun. 12, 2008

(54) Inhibition of inflammatory cytokine production by stimulation of brain muscarinic receptors

(76) Inventors: Svetlana M. Ivanova, Astoria, NY (US); Kevin J. Tracey, Old Greenwich, CT (US)

Correspondence Address: SHAY GLENN LLP, 2755 CAMPUS DRIVE, SUITE 210, SAN MATEO, CA, 94403, US

(21) Appl. No.: 11/807,493

(22) Filed: May 29, 2007

Related U.S. Application Data
(63) Continuation of application No. 10/375,696, filed on Feb. 26, 2003, now abandoned.
(60) Provisional application No. 60/360,082, filed on Feb. 26, 2002.

(51) Int. Cl. A61N 1/00

(52) U.S. Cl. 607/2

(57) ABSTRACT

Methods for inhibiting pro-inflammatory cytokine release or inflammation in a vertebrate are provided. The methods comprise activating a brain muscarinic receptor of the vertebrate, or directly stimulating a vagus nerve pathway in the brain of the vertebrate. Also provided are methods for conditioning a vertebrate to inhibit the release of a pro-inflammatory cytokine or reduce inflammation in the vertebrate upon experiencing a sensory stimulus. The methods comprise (a) activating a muscarinic brain receptor or directly stimulating the vagus nerve pathway in the brain of the vertebrate and providing the sensory stimulus to the vertebrate within a time period sufficient to create an association between the stimulus and the activation of the brain muscarinic receptor; and (b) repeating step (a) at sufficient time intervals and duration to reinforce the association sufficiently for the inflammation to be reduced by the sensory stimulus alone.

### RELATED APPLICATION

This application is a continuation of U.S. application Ser. No. 10/375,696, filed Feb. 26, 2003, which claims the benefit of U.S. Provisional Application No. 60/360,082, filed Feb. 26, 2002. The entire teachings of the above application are incorporated herein by reference.

### GOVERNMENT SUPPORT

The invention was supported, in whole or in part, by a grant RO1 GM057226 from the National Institutes of Health and by grants N00178-01-C-3058 and N66001-01-1-8970 from the Department of Defense. The Government has certain rights in the invention.

### BACKGROUND OF THE INVENTION

### Field of the Invention

The present invention generally relates to methods of reducing inflammation. More specifically, the invention relates to methods for reducing inflammation caused by proinflammatory cytokines or an inflammatory cytokine cascade.

Vertebrates achieve internal homeostasis during infection or injury by balancing the activities of proinflammatory and anti-inflammatory pathways. However, in many disease conditions, this internal homeostasis becomes out of balance. For example, endotoxin (lipopolysaccharide, LPS), produced by all Gram-negative bacteria, activates macrophages to release cytokines that are potentially lethal to the host (Tracey et al., 1986; Dinarello, 1994; Wang, H., et al., 1999; Nathan, 1987).

Inflammation and other deleterious conditions (such as septic shock caused by endotoxin exposure) are often induced by proinflammatory cytokines, such as tumor necrosis factor (TNF; also known as TNFα or cachectin), interleukin (IL)-1α, IL-1β, IL-6, IL-8, IL-18, interferon-γ, platelet-activating factor (PAF), macrophage migration inhibitory factor (MIF), and other compounds (Thompson, 1998). Certain other compounds, for example, high mobility group protein 1 (HMG-B1), are induced during various conditions, such as sepsis, and can also serve as proinflammatory cytokines (WO 00/47104). These proinflammatory cytokines are produced by several different cell types, most importantly immune cells (for example, monocytes, macrophages, and neutrophils), but also non-immune cells such as fibroblasts, osteoblasts, smooth muscle cells, epithelial cells, and neurons (Zhang and Tracey, 1998). Proinflammatory cytokines contribute to various disorders, notably sepsis, through their release during an inflammatory cytokine cascade.

Inflammatory cytokine cascades contribute to deleterious characteristics of numerous disorders. These deleterious characteristics include inflammation and apoptosis (Pulkki, 1997). Disorders where inflammatory cytokine cascades are involved at least in part, include, without limitation, diseases involving the gastrointestinal tract and associated tissues (such as appendicitis, peptic, gastric and duodenal ulcers, peritonitis, pancreatitis, ulcerative, pseudomembranous, acute and ischemic colitis, inflammatory bowel disease, diverticulitis, epiglottitis, achalasia, cholangitis, coeliac disease, cholecystitis, hepatitis, Crohn's disease, enteritis, and Whipple's disease); systemic or local inflammatory diseases and conditions (such as asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, and sarcoidosis); diseases involving the urogenital system and associated tissues (such as septic abortion, epididymitis, vaginitis, prostatitis, and urethritis); diseases involving the respiratory system and associated tissues (such as bronchitis, emphysema, rhinitis, cystic fibrosis, adult respiratory distress syndrome, pneumonitis, pneumoultramicroscopic silicovolcanoconiosis, alveolitis, bronchiolitis, pharyngitis, pleurisy, and sinusitis); diseases arising from infection by various viruses (such as influenza, respiratory syncytial virus, HIV, hepatitis B virus, hepatitis C virus, and herpes), bacteria (such as disseminated bacteremia, Dengue fever), fungi (such as candidiasis) and protozoal and multicellular parasites (such as malaria, filariasis, amebiasis, and hydatid cysts); dermatological diseases and conditions of the skin (such as burns, dermatitis, dermatomyositis, sunburn, urticaria warts, and wheals); diseases involving the cardiovascular system and associated tissues (such as vasculitis, angiitis, endocarditis, arteritis, atherosclerosis, thrombophlebitis, pericarditis, myocarditis, myocardial ischemia, congestive heart failure, periarteritis nodosa, and rheumatic fever); diseases involving the central or peripheral nervous system and associated tissues (such as Alzheimer's disease, meningitis, encephalitis, multiple sclerosis, cerebral infarction, cerebral embolism, Guillame-Barre syndrome, neuritis, neuralgia, spinal cord injury, paralysis, and uveitis); diseases of the bones, joints, muscles, and connective tissues (such as the various arthritis and arthralgias, osteomyelitis, fasciitis, Paget's disease, gout, periodontal disease, rheumatoid arthritis, and synovitis); other autoimmune and inflammatory disorders (such as myasthenia gravis, thyroiditis, systemic lupus erythematosus, Goodpasture's syndrome, Behcets's syndrome, allograft rejection, graft-versus-host disease, Type I diabetes, Berger's disease, and Retier's syndrome); as well as various cancers, tumors and proliferative disorders (such as Hodgkins disease); and, in any case the inflammatory or immune host response to any primary disease (see, e.g., Gattorno et al., 2000; Yeh and Schuster, 1999; McGuinness et al., 2000; Hsu et al., 1999; Jander and Stoll, 2001; Kanai et al., 2001; Prystowsky and Rege, 1997; Kimmings et al., 2000; Hirano, T., 1999; Lee et al., 1995; Waserman et al., 2000; Watanabe et al., 1997; Katagiri, et al., 1997; Bumgardner, and Orosz, 1999; Dibbs, et al., 1999; Blackwell and Christman, 1996; Blum and Miller, 1998; Carteron, 2000; Fox, 2000; Hommes and van Deventer, 2000; Gracie et al., 1999; Rayner et al. 2000).

Tumor necrosis factor is known to be a major pro-inflammatory cytokine mediator of various acute and chronic inflammatory diseases, e.g., gram negative bacterial sepsis, multi-system organ failure (MSOF), circulatory collapse and death. The primary source of circulating TNF following a septic challenge is the liver. Thus, rats subjected to two-thirds hepatectomy produce 64% less TNF after endotoxin, as compared to sham controls (Kumins et al., 1996).

Direct production of TNF by cardiac muscle also appears to play a major role in septic myocardial depression. Myocytes respond to stress by primary production of TNF, as well as by increasing TNF receptors (Irwin et al., 1999). TNF, either produced locally in the heart, or originating from other sources, causes myocyte apoptosis and thrombosis (Song et al., 2000). TNF has been implicated in various cardiac disorders including cardiac failure secondary to septic cardiomyopathy, bi-ventricular dysfunction, and pulmonary edema. TNF can also have a direct negative inotropic effect on cardiac function.

Vertebrates respond to inflammation caused by inflammatory cytokine cascades in part through humoral mechanisms of the central nervous system (activation of the hypothalamus-pituitary adrenal [HPA] axis), by means of vagal nerve activation, and by means of peripheral anti-inflammatory cytokine production (e.g., IL-10 production). This response has been characterized in detail with respect to systemic humoral response mechanisms during inflammatory responses to endotoxin (Besedovsky et al., 1986; Woiciechowsky et al., 1998; Hu et al., 1991; Lipton and Catania, 1997).

The vagus nerve is a critical cranial nerve in modulating whole body homeostasis, including, inter alia, inflammatory regulation through both afferent and efferent signaling. Vagus nerve fibers reach multiple internal organs, such as the trachea/bronchi, abdominal blood vessels, kidneys, small and large intestine, adrenals, liver, and heart. The paws of an animal have also been shown to receive vagus nerve innervation via nerve fibers traveling along the blood vessels, as well as nerve fibers in sweat glands, etc.

In one set of responses, afferent vagus nerve fibers are activated by endotoxin or cytokines, stimulating the release of humoral anti-inflammatory responses through glucocorticoid hormone release (Watkins and Maier, 1999; Sternberg, 1997; Scheinman et al, 1995). Cytokines or endotoxin can stimulate the afferent vagus nerve, which in turn signals a number of critical brain nuclei, and leads to activation of the HPA anti-inflammatory responses and down-regulation of endotoxemia and cytokinemia (Gaykema et al., 1995; Fleshner et al., 1998; Watkins et al., 1995; Romanovsky et al., 1997). Similarly, direct efferent vagus nerve stimulation (VNS) in rats prevents shock secondary to an induced endotoxic challenge, by decreasing TNF synthesis in the liver (see U.S. patent application Ser. No. 09/855,446, the teachings of which are incorporated herein by reference). The efferent vagus nerve can also be stimulated to achieve immunosuppression by pharmacological means. For example, the anti-inflammatory pharmacological agent CNI-1493, when administered peripherally, has the ability to cross the blood-brain barrier, and activate the efferent vagus nerve through a central mechanism of action, thus mediating peripheral immunosuppression, with anti-inflammatory effects (Borovikova et al., 2000). Intracerebroventricular administration of CNI-1493 is also an effective anti-inflammatory treatment (Id.)

The effect of direct stimulation of brain cholinergic agonists on inflammation was evaluated in Bhattacharya et al. (1991). In those studies, direct administration of high doses of muscarinic agonists caused augmentation of carrageenan-induced paw edema. Although low doses of the muscarine agonist carbachol caused attenuation of paw edema, the authors concluded that, overall, muscarinic agonist treatment of the brain caused augmentation of paw edema. There was also no suggestion in that paper that the muscarinic agonist could be useful in reducing inflammation.

### Conditioning of the Immune System.

Conditioning is a method of training an animal by which a perceptible neutral stimulus is temporarily associated with a physiological stimulus so that the animal will ultimately respond to the neutral stimulus as if it were the physiological stimulus. Pavlov, for instance, trained dogs to respond with salivation to the ringing of a bell following prior experiments where the dogs were prescribed a food stimulus (associated with salivation) simultaneously with a ringing bell stimulus.

Elmer Green (1969) proposed that perception elicits mental and emotional responses, generating limbic, hypothalamic, and pituitary responses that bring about physiological changes. Ader and Cohen (1982) further extended the scope of conditioning to the immune system. They showed that rats could be conditioned to respond to a neutral stimulus, saccharin, with a decreased immune response after having been repeatedly and simultaneously exposed to cyclophosphamide, an immunosuppressive drug. The observed effects extended to both humoral immunity (i.e., antibody production) as well as to cellular immunity (i.e., graft vs. host response) (Ader and Cohen, 1975; Cohen et al., 1979; Ader and Cohen, 1982; Ader and Cohen, 1992).

Human studies have also linked immune dysregulation with psychological disease (Cohen et al., 2001). Additionally, hypnosis (Wyler-Harper et al., 1994; Fox et al., 1999) and biofeedback (Peavey et al., 1985) has been found to be effective in modulating the immune response.

### SUMMARY OF THE INVENTION

Accordingly, the inventors have succeeded in discovering that pro-inflammatory cytokine release in vertebrates, and the associated inflammatory responses, can be inhibited by activating brain muscarinic receptors. Further, the inventors have discovered that this anti-inflammatory response can be conditioned by repeated association of a sensory stimulus with activation of brain muscarinic receptors. These discoveries enable novel methods for inhibiting pro-inflammatory cytokine release and inflammation.

Thus, in one aspect, the present invention is directed to methods of inhibiting release of a pro-inflammatory cytokine in a vertebrate. The method comprises activating a brain muscarinic receptor in the vertebrate.

The present invention is also directed to methods of inhibiting release of a pro-inflammatory cytokine in a vertebrate. The method comprises directly stimulating a vagus nerve pathway in the brain of the vertebrate.

In additional embodiments, the invention is directed to methods of treating an inflammatory disease in a vertebrate. The methods comprise activating a brain muscarinic receptor in the vertebrate.

The invention is additionally directed to methods of treating an inflammatory disease in a vertebrate. The methods comprise directly stimulating a vagus nerve pathway in the brain of the vertebrate.

In another aspect, the present invention is directed to methods of inhibiting apoptosis of a cardiac myocyte in a vertebrate at risk for cardiac myocyte apoptosis. The methods comprise activating a brain muscarinic receptor in the vertebrate.

The present invention is also directed to methods of inhibiting apoptosis of a cardiac myocyte in a vertebrate at risk for cardiac myocyte apoptosis. The methods comprise directly stimulating a vagus nerve pathway in the brain of the vertebrate.

In additional embodiments, the present invention is directed to methods of conditioning a vertebrate to inhibit the release of a pro-inflammatory cytokine upon experiencing a sensory stimulus. The methods comprise the following steps:

(a) activating a brain muscarinic receptor in the vertebrate and providing the sensory stimulus to the vertebrate within a time period sufficient to create an association between the stimulus and the activation of the brain muscarinic receptor; and

(b) repeating step (a) at sufficient time intervals and duration to reinforce the association sufficiently for the pro-inflammatory cytokine release to be inhibited by the sensory stimulus alone.

The invention is also directed to methods of conditioning a vertebrate to inhibit the release of a pro-inflammatory cytokine upon experiencing a sensory stimulus. The methods comprise the following steps:

(a) directly stimulating a vagus nerve pathway in the brain of the vertebrate and providing the sensory stimulus to the vertebrate within a time period sufficient to create an association between the stimulus and the stimulation of a vagus nerve pathway; and

(b) repeating step (a) at sufficient time intervals and duration to reinforce the association sufficiently for the pro-inflammatory cytokine release to be inhibited by the sensory stimulus alone.

The invention is additionally directed to methods of conditioning a vertebrate to reduce inflammation in the vertebrate upon experiencing a sensory stimulus. The methods comprise the following steps:

(a) activating a brain muscarinic receptor in the vertebrate and providing the sensory stimulus to the vertebrate within a time period sufficient to create an association between the stimulus and the activation of the brain muscarinic receptor; and

(b) repeating step (a) at sufficient time intervals and duration to reinforce the association sufficiently for the inflammation to be reduced by the sensory stimulus alone.

Additionally, the present invention is directed to methods of conditioning a vertebrate to reduce inflammation in the vertebrate upon experiencing a sensory stimulus. The methods comprise the following steps:

(a) directly stimulating a vagus nerve pathway in the brain of the vertebrate and providing the sensory stimulus to the vertebrate within a time period sufficient to create an association between the stimulus and the activation of the brain muscarinic receptor; and

(b) repeating step (a) at sufficient time intervals and duration to reinforce the association sufficiently for the inflammation to be reduced by the sensory stimulus alone.

### BRIEF DESCRIPTION OF THE DRAWINGS

FIG. 1 is a graph summarizing the results of experiments showing that intracerebroventricular administration of CNI-1493 significantly inhibits LPS-induced release of TNF, and that atropine (ATR) reverses the effect.

FIG. 2 is a graph summarizing the results of experiments showing that intracerebroventricular administration of nicotine or prozak has no effect on LPS-induced release of TNF.

FIG. 3 is a graph summarizing the results of experiments showing that intracerebroventricular administration of CNI-1493 significantly inhibits carageenan-induced paw edema, and that atropine (ATR) reverses the effect.

FIG. 4 is a graph summarizing the results of experiments showing that intracerebroventricular administration of muscarine significantly inhibits carrageenan-induced paw edema in a dose-dependent manner.

FIG. 5 is a graph summarizing the results of experiments showing that vagotomy abrogates the inhibitory effects of intracerebroventricular (i.c.v.) administration of muscarine on carrageenan-induced paw edema.

FIG. 6 is a graph summarizing the results of experiments showing that intracerebroventricular administration of the M1 agonist McN-A-343 or the M4 agonist MT-3 significantly inhibits carrageenan-induced paw edema.

FIG. 7 is a graph summarizing the results of experiments showing that intracerebroventricular (i.c.v.) administration of the M1 agonist McN-A-343 is significantly more potent in inhibiting carrageenan-induced paw edema as compared to intraperitoneal (i.p.) administration.

FIG. 8 is a graph summarizing the results of experiments showing that conditioning animals by associating intraperitoneal CNI-1493 administration with bell ringing allowed the inhibition of LPS-induced TNF release by bell ringing without CNI-1493 administration.

FIG. 9A is a graph summarizing the results of the effect of intracerebroventricular (i.c.v.) administration of no muscarine (control), or muscarine at 0.005 μg/kg body weight, 0.5 μg/kg body weight, 5.0 μg/kg body weight, or 50 μg/kg body weight on LPS-induced TNF production (TNF concentration (pg/ml)) in the serum of rats. R indicates the number of rats per test condition.

FIG. 9B is a graph summarizing the results of the effect of intracerebroventricular (i.c.v.) administration of no muscarine (control), or muscarine at 0.005 μg/kg body weight, 0.5 μg/kg body weight, 5.0 μg/kg body weight, or 50 μg/kg body weight on LPS-induced TNF production (TNF concentration (ng/g protein)) in the heart tissues of rats. R indicates the number of rats per test condition.

FIG. 9C is a graph summarizing the results of the effect of intracerebroventricular (i.c.v.) administration of no muscarine (control), or muscarine at 0.005 μg/kg body weight, 0.5 μg/kg body weight, 5.0 μg/kg body weight, or 50 μg/kg body weight on LPS-induced TNF production (TNF concentration (ng/g protein)) in the spleens of rats. R indicates the number of rats per test condition.

FIG. 10A is a graph summarizing the results of the effect of intravenous (i.v.) administration of no muscarine (control), or muscarine at 0.05 μg/kg body weight, 0.5 μg/kg body weight, or 5.0 μg/kg body weight on LPS-induced TNF production (TNF concentration (pg/ml)) in the serum of rats. R indicates the number of rats per test condition.

FIG. 10B is a graph summarizing the results of the effect of intravenous (i.v.) administration of no muscarine (control), or muscarine at 0.05 μg/kg body weight, 0.5 μg/kg body weight, or 5.0 μg/kg body weight on LPS-induced TNF production (TNF concentration (ng/g protein)) in the livers of rats. R indicates the number of rats per test condition.

FIG. 10C is a graph summarizing the results of the effect of intravenous (i.v.) administration of no muscarine (control), or muscarine at 0.05 μg/kg body weight, 0.5 μg/kg body weight, or 5.0 μg/kg body weight on LPS-induced TNF production (TNF concentration (ng/g protein)) in the spleens of rats. R indicates the number of rats per test condition.

FIG. 10D is a graph summarizing the results of the effect of intravenous (i.v.) administration of no muscarine (control), or muscarine at 0.05 μg/kg body weight, 0.5 μg/kg body weight, or 5.0 μg/kg body weight on LPS-induced TNF production (TNF concentration (ng/g protein)) in the heart tissues of rats. R indicates the number of rats per test condition.

### DETAILED DESCRIPTION OF THE INVENTION

The present invention is based on the discovery that activation of vertebrate brain muscarinic receptors causes an inhibition of the release of various pro-inflammatory cytokines in the periphery, which in turn causes a reduction of peripheral inflammation. This reduction of peripheral inflammation can be achieved by muscarinic agonist treatment or by exposure to an external sensory stimulus after Pavlovian conditioning by prior repeated association of the stimulus with the muscarinic agonist treatment. The inhibition of pro-inflammatory cytokine release and the reduction of peripheral inflammation is vagus nerve-dependent and can also be reduced by direct stimulation of the vagus nerve in the brain. These discoveries enable the treatment of various inflammatory conditions in novel ways.

As used herein, a cytokine is a soluble protein or peptide which is naturally produced by vertebrate cells and which act in vivo as humoral regulators at micro- to picomolar concentrations. Cytokines can, either under normal or pathological conditions, modulate the functional activities of individual cells and tissues. A pro-inflammatory cytokine is a cytokine that is capable of causing any of the following physiological reactions associated with inflammation: vasodilatation, hyperemia, increased permeability of vessels with associated edema, accumulation of granulocytes and mononuclear phagocytes, or deposition of fibrin. In some cases, the pro-inflammatory cytokine can also cause apoptosis, such as in chronic heart failure, where TNF has been shown to stimulate cardiomyocyte apoptosis (Pulkki, 1997; Tsutsui et al., 2000). Nonlimiting examples of pro-inflammatory cytokines are tumor necrosis factor (TNF), interleukin (IL)-1α, IL-1β, IL-6, IL-8, IL-18, interferon-γ, HMG-B1, platelet-activating factor (PAF), and macrophage migration inhibitory factor (MIF). In preferred embodiments of the invention, the pro-inflammatory cytokine that is inhibited by cholinergic agonist treatment is TNF, IL-1, IL-6, or IL-18, because these cytokines are produced by macrophages and mediate deleterious conditions for many important disorders, for example, endotoxic shock, asthma, rheumatoid arthritis, inflammatory bile disease, heart failure, and allograft rejection. In most preferred embodiments, the pro-inflammatory cytokine is TNF.

Pro-inflammatory cytokines are to be distinguished from anti-inflammatory cytokines, such as IL-4, IL-10, and IL-13, which tend to inhibit inflammation. In preferred embodiments, release of anti-inflammatory cytokines is not inhibited by cholinergic agonists.

In many instances, pro-inflammatory cytokines are produced in an inflammatory cytokine cascade, defined herein as an in vivo release of at least one pro-inflammatory cytokine in a vertebrate, wherein the cytokine release affects a physiological condition of the vertebrate. Thus, an inflammatory cytokine cascade is inhibited in embodiments of the invention where pro-inflammatory cytokine release causes a deleterious physiological condition.

Nonlimiting examples of diseases characterized by the presence of deleterious physiological conditions at least partially mediated by pro-inflammatory cytokine release are appendicitis, peptic, gastric or duodenal ulcers, peritonitis, pancreatitis, ulcerative, pseudomembranous, acute or ischemic colitis, inflammatory bowel disease, diverticulitis, epiglottitis, achalasia, cholangitis, cholecystitis, hepatitis, Crohn's disease, enteritis, Whipple's disease, asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, pneumoultramicroscopic silicovolcanoconiosis, alveolitis, bronchiolitis, pharyngitis, pleurisy, sinusitis, influenza, respiratory syncytial virus, herpes, disseminated bacteremia, Dengue fever, candidiasis, malaria, filariasis, amebiasis, hydatid cysts, burns, dermatitis, dermatomyositis, sunburn, urticaria, warts, wheals, vasculitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritis, arthralgias, osteomyelitis, fasciitis, Paget's disease, gout, periodontal disease, rheumatoid arthritis, synovitis, myasthenia gravis, thyroiditis, systemic lupus erythematosus, Goodpasture's syndrome, Behcets's syndrome, allograft rejection, graft-versus-host disease, Type I diabetes, ankylosing spondylitis, Berger's disease, Retier's syndrome, and Hodgkins disease. Additional examples of conditions mediated by pro-inflammatory cytokine release include shock, for example, hemorrhagic shock, chronic obstructive pulmonary disease (COPD) and psoriasis.

Any vertebrate cell that produces pro-inflammatory cytokines is useful for the practice of the invention. Nonlimiting examples are monocytes, macrophages, any cells resident in the liver that make, transport, or concentrate pro-inflammatory cytokines including Kupffer cells and biliary endothelial cells, neutrophils, epithelial cells, osteoblasts, fibroblasts, hepatocytes, muscle cells including smooth muscle cells and cardiac myocytes, and neurons. In preferred embodiments, the cell is a macrophage, Kupffer cell, monocyte, biliary endothelial cell, hepatocyte, or cardiac myocyte.

As used herein, a cholinergic agonist is a compound that binds to cholinergic receptors on cells. The skilled artisan can determine whether any particular compound is a cholinergic agonist by any of several well known methods.

When referring to the effect of the cholinergic agonist on release of pro-inflammatory cytokines or an inflammatory cytokine cascade, or the effect of vagus nerve stimulation on an inflammatory cytokine cascade, the use of the terms “inhibit” or “decrease” encompasses at least a small but measurable reduction in pro-inflammatory cytokine release. In preferred embodiments, the release of the pro-inflammatory cytokine is inhibited by at least 20% over non-treated controls; in more preferred embodiments, the inhibition is at least 50%; in still more preferred embodiments, the inhibition is at least 70%, and in the most preferred embodiments, the inhibition is at least 80%. Such reductions in pro-inflammatory cytokine release are capable of reducing the deleterious effects of an inflammatory cytokine cascade.

Accordingly, in some embodiments, the present invention is directed to methods of inhibiting the release of a pro-inflammatory cytokine in a vertebrate. The methods comprise activating a brain muscarinic receptor in the vertebrate. In preferred embodiments, the pro-inflammatory cytokine is tumor necrosis factor (TNF), interleukin (IL)-1β, IL-6, IL-18, HMG-B1, MIP-1α, MIP-1β, MIF, interferon-γ, or PAF. In more preferred embodiments, the pro-inflammatory cytokine is selected from the group consisting of tumor necrosis factor (TNF), interleukin (IL)-1β, IL-6, IL-18, and HMG-B1. In the most preferred embodiments, the pro-inflammatory cytokine is TNF.

These methods are useful for preventing the release of pro-inflammatory cytokines in any vertebrate. In preferred embodiments, the vertebrate is a mammal. In particularly preferred embodiments, the vertebrate is a human. The vertebrate is preferably a patient suffering from, or at risk for, a condition mediated by an inflammatory cytokine cascade. As used herein, a patient can be any vertebrate individual from a species that has a vagus nerve. Preferably, the condition is appendicitis, peptic, gastric and duodenal ulcers, peritonitis, pancreatitis, ulcerative, pseudomembranous, acute and ischemic colitis, inflammatory bowel disease, diverticulitis, epiglottitis, achalasia, cholangitis, cholecystitis, hepatitis, Crohn's disease, enteritis, Whipple's disease, asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, pneumoultramicroscopic silicovolcanoconiosis, alveolitis, bronchiolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasculitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritis, arthralgias, osteomyelitis, fasciitis, Paget's disease, gout, periodontal disease, rheumatoid arthritis, synovitis, myasthenia gravis, thyroiditis, systemic lupus erythematosus, Goodpasture's syndrome, Behcets's syndrome, allograft rejection, graft-versus-host disease, Type I diabetes, ankylosing spondylitis, Berger's disease, Retier's syndrome, and Hodgkins disease. More preferably, the condition is appendicitis, peptic, gastric and duodenal ulcers, peritonitis, pancreatitis, ulcerative, pseudomembranous, acute and ischemic colitis, inflammatory bowel disease, hepatitis, Crohn's disease, asthma, allergy, anaphylactic shock, organ ischemia, reperfusion injury, organ necrosis, hay fever, sepsis, septicemia, endotoxic shock, cachexia, septic abortion, disseminated bacteremia, burns, Alzheimer's disease, coeliac disease, congestive heart failure, adult respiratory distress syndrome, cerebral infarction, cerebral embolism, spinal cord injury, multiple sclerosis, paralysis, allograft rejection and graft-versus-host disease. In most preferred embodiments, the condition is endotoxic shock.

These methods can be used to prevent release of pro-inflammatory cytokines in the brain or any peripheral organ served by the vagus nerve. Preferred examples include the liver, which makes pro-inflammatory cytokines involved in systemic inflammatory cascades such as endotoxic shock. Another preferred peripheral organ is the heart, since it is known that cardiac myocytes release pro-inflammatory cytokines implicated in myocyte apoptosis and thrombosis.

The preferred brain muscarinic receptors to be activated in these methods are the M1, M2, and M4 receptors, since these receptors cause the strongest effect in inhibiting release of pro-inflammatory cytokines. See Example 2. Thus, in embodiments that utilize a muscarinic agonist to activate the muscarinic receptor, one that activates the M1, M2, and/or M4 receptors are particularly preferred. Nonlimiting examples of preferred muscarinic agonists useful for these methods include muscarine, McN-A-343, and MT-3. In one embodiment, the muscarinic agonist is not N,N′-bis(3,5-diacetylphenyl) decanediamide tetrakis (amidinohydrazone) tetrahydrochloride (CNI-1493). In another embodiment, the muscarinic agonist is not a CNI-1493 compound. As used herein, “a CNI-1493 compound” means an aromatic guanylhydrazone (“Ghy”, more properly termed amidinohydrazone, i.e., NH₂(CNH(—NH═) compound having the formula:

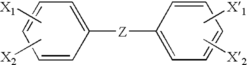

wherein X₂=GhyCH—, GhyCCH₃— or H—; X₁, X′₁ and X′₂ independently=GhyCH— or GhyCCH₃—; Z=—NH(CO)NH—, —(C₆H₄)—, —(C₅H₃)— or -A-(CH₂)ₙ-A-, n=2-10, which is unsubstituted, mono- or di-C-methyl substituted, or a mono or di-unsaturated derivative thereof; and A, independently, ═—NH(CO)—, —NH(CO)NH—, —NH— or —O— and salts thereof. GhyCH—═NH₂(CNH)—NH—N═CH—, and GhyCCH₃—═NH₂(CNH)—NH—N═CCH₃—. A preferred embodiment includes those compounds wherein A is a single functionality. Also included are compounds having the same formula wherein X₁ and X₂═H; X′₁ and X′₂ independently=GhyCH— or GhyCCH₃—; Z=-A-(CH₂)ₙ-A-, n=3-8; and A=—NH(CO)— or —NH(CO)NH—, and salts thereof. Also included are compounds wherein X₁ and X₂═H; X′₁ and X′₂ independently=GhyCH— or GhyCCH₃— and Z=—O—(CH₂)₂—O—.

Further examples of CNI-1493 compounds include: compounds of the above formula wherein: X₂=GhyCH—, GhyCCH₃— or H—; X₁, X′₁ and X′₂=GhyCH— or GhyCCH₃—; and Z=—O—(CH₂)ₙ—O—, n=2-10 and salts thereof; and the related compounds wherein, when X₂ is other than H, X₂ is meta or para to X₁ and wherein X′₂ is meta or para to X′₁. A compound having the above formula wherein: X₂=GhyCH, GhyCCH₃ or H; X₁, X′₁ and X′₂, =GhyCH— or GhyCCH₃—; and Z=—NH— (C═O)—NH— and salts thereof; and the related genus wherein, when X₂ is other than H, X₂ is meta or para to X₁ and wherein X′₂ is meta or para to X′₁.

A “CNI-1493 compound” also means an aromatic guanylhydrazone compound having the formula:

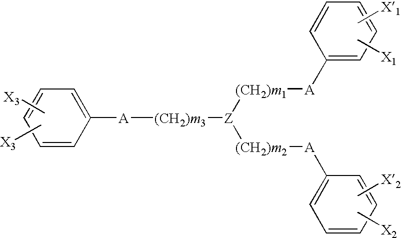

wherein, X₁, X₂ and X₃, independently=GhyCH— or GhyCCH₃—; X′₁, X₂ and X′₃, independently=H, GhyCH— or GhyCCH₃—; Z=(C₆H₃), when m₁, m₂, m₃=0 or Z=N, when, independently, m₁, m₂, m₃=2-6; and A=—NH(CO)—, —NH(CO)NH—, —NH— or —O— and salts thereof. Further examples of CNI-1493 include the genus wherein when any of X′₁, X₂ and X′₃ are other than H, then the corresponding substituent of the group consisting of X₁, X₂ and X₃ is meta or para to X′₁, X₂ and X′₃, respectively; the genus wherein, m1, m2, m₃=0 and A=—NH(CO)—; and the genus wherein m₁, m₂, m₃=2-6 and A=—NH(CO)NH—. Examples of CNI-1493 and methods for making such compounds are described in U.S. Pat. No. 5,854,289 (the teachings of which are incorporared herein by reference). In a preferred embodiment, the CNI-1493 compound is N,N′-bis(3,5-diacetylphenyl) decanediamide tetrakis (amidinohydrazone) tetrahydrochloride (also known as CNI-1493), which can be made by combining N,N′-bis(3,5-diacetylphenyl)decanediamide (0.65 g), aminoguanidine hydrochloride (0.691 g), and aminoguanidine dihydrochloride (0.01 g) and heating in 91% ethanol (5.5 mL) for 18 hr, followed by cooling and filtration. The synthesis results in a compound having a melting point of 323° C.-324° C. The composition can be formulated in a physiologically acceptable carrier.

Activation of brain muscarinic receptors can thus be achieved by treatment with a muscarinic agonist. As used herein, a muscarinic agonist is an agonist that can bind to a muscarine receptor. In an embodiment, the muscarinic agonist can bind to other receptor type(s) in addition to the muscarine receptor, for example, another cholinergic receptor. An example of such a muscarinic agonist is acetylcholine. In another embodiment, the muscarinic agonist binds muscarine receptor(s) with greater affinity than other cholinergic receptors, e.g., nicotinic receptors (e.g., with at least 10% greater affinity, 20% greater affinity 50% greater affinity, 75% greater affinity 90% greater affinity or 95% greater affinity). In one embodiment the muscarinic agonist is selective for an M1, M2, or M4 receptor. As used herein, an agonist that is “selective” for an M1, M2, or M4 receptor is an agonist that binds to an M1, M2, and/or M4 receptor with greater affinity than it binds to one, two, or more other receptors, for example, one or more other muscarinic receptors (e.g., M3 or M5 muscarinic receptors), or one or more other cholinergic receptors. In an embodiment, the agonist binds with at least 10% greater affinity, 20% greater affinity 50% greater affinity, 75% greater affinity 90% greater affinity or 95% greater affinity than it binds to receptors other than an M1, M2, and/or M4 receptor. Binding affinities can be determined as described herein or using other receptor binding assays known to one of skill in the art. In one embodiment, the brain muscarinic receptor is activated with a sufficient amount of muscarinic agonist or at a sufficient level to inhibit release of a pro-inflammatory cytokine from a vertebrate cell.

The muscarinic agonist can be administered to the brain muscarinic receptors by intracerebroventricular injection. Alternatively, the muscarinic agonist can be administered orally, parenterally, intranasally, vaginally, rectally, lingually, sublingually, bucally, intrabuccaly, or transdermally to the patient, provided the muscarinic agonist can cross the blood-brain barrier.

The route of administration of the muscarinic agonist can depend on the condition to be treated. For example, intravenous injection may be preferred for treatment of a systemic disorder such as septic shock, and oral administration may be preferred to treat a gastrointestinal disorder such as a gastric ulcer. The route of administration and the dosage of the cholinergic agonist to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms.

Muscarinic agonist compositions useful for the present invention can be administered parenterally such as, for example, by intravenous, intramuscular, intrathecal, or subcutaneous injection. Parenteral administration can be accomplished by incorporating the muscarinic agonist compositions of the present invention into a solution or suspension. Such solutions or suspensions may also include sterile diluents such as water for injection, saline solution, fixed oils, polyethylene glycols, glycerine, propylene glycol, or other synthetic solvents. Parenteral formulations may also include antibacterial agents such as, for example, benzyl alcohol, or methyl parabens, antioxidants such as, for example, ascorbic acid or sodium bisulfite and chelating agents such as EDTA. Buffers such as acetates, citrates, or phosphates and agents for the adjustment of tonicity such as sodium chloride or dextrose may also be added. The parenteral preparation can be enclosed in ampules, disposable syringes, or multiple dose vials made of glass or plastic.

Rectal administration includes administering the pharmaceutical compositions into the rectum or large intestine. This can be accomplished using suppositories or enemas. Suppository formulations can be made by methods known in the art. For example, suppository formulations can be prepared by heating glycerin to about 120° C., dissolving the cholinergic agonist in the glycerin, mixing the heated glycerin after which purified water may be added, and pouring the hot mixture into a suppository mold.

Transdermal administration includes percutaneous absorption of the cholinergic agonist through the skin. Transdermal formulations include patches, ointments, creams, gels, salves, and the like.

The present invention includes nasally administering to the vertebrate a therapeutically effective amount of the muscarinic agonist. As used herein, nasal administration includes administering the cholinergic agonist to the mucous membranes of the nasal passage or nasal cavity of the patient. As used herein, pharmaceutical compositions for nasal administration of a cholinergic agonist include therapeutically effective amounts of the agonist prepared by well-known methods to be administered, for example, as a nasal spray, nasal drop, suspension, gel, ointment, cream, or powder. Administration of the cholinergic agonist may also take place using a nasal tampon, or nasal sponge.

Accordingly, muscarinic agonist compositions designed for oral, lingual, sublingual, buccal and intrabuccal administration can be made without undue experimentation by means well known in the art, for example, with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums, and the like.

Tablets, pills, capsules, troches, and the like may also contain binders, recipients, disintegrating agent, lubricants, sweetening agents, and flavoring agents. Some examples of binders include microcrystalline cellulose, gum tragacanth, or gelatin. Examples of excipients include starch or lactose. Some examples of disintegrating agents include alginic acid, corn starch, and the like. Examples of lubricants include magnesium stearate or potassium stearate. An example of a glidant is colloidal silicon dioxide. Some examples of sweetening agents include sucrose, saccharin, and the like. Examples of flavoring agents include peppermint, methyl salicylate, orange flavoring, and the like. Materials used in preparing these various compositions should be pharmaceutically pure and nontoxic in the amounts used.

As previously discussed, the effect of activation of a brain muscarinic receptor on inhibiting the release of pro-inflammatory cytokines in the periphery is established herein to be dependent on an intact vagus nerve. Without being limited to any particular mechanism, the inventors believe that brain muscarinic receptor activation stimulates the vagus nerve pathway, and this stimulation causes the inhibition of pro-inflammatory cytokine release. This stimulation of the brain vagus nerve pathway is “upstream” in the vagus nerve pathway from the previously established effect of stimulation of peripheral vagus nerves on inhibiting pro-inflammatory cytokine release (Borovikova et al., 2000a; see also U.S. patent application Ser. No. 09/855,446). Based on the determination that an intact vagus pathway is required for the inhibition of pro-inflammatory cytokine release effected by brain muscarinic agonist activation, as established herein, it is clear that pro-inflammatory cytokines can be inhibited by directly stimulating a vagus nerve pathway in the brain. In one embodiment, the vagus nerve pathway is stimulated at a sufficient level to inhibit release of a pro-inflammatory cytokine from a vertebrate cell.

Accordingly, some embodiments of the present invention are directed to methods of inhibiting release of a pro-inflammatory cytokine in a vertebrate. The methods comprise directly stimulating the vagus nerve pathway in the brain of the vertebrate. In these methods the vagus nerve pathway can be stimulated by any known method. Nonlimiting examples include mechanical means such as a needle, ultrasound, or vibration; pharmacological or chemical stimulation, any electromagnetic radiation such as infrared, visible or ultraviolet light; heat, or any other energy source. In preferred embodiments, the vagus nerve is stimulated electrically, for example, with a commercial deep brain stimulator, such as the Medtronic SOLETRA device, which is currently in use for the treatment of Parkinson's disease, etc. In preferred embodiments, the vagus nerve pathway is stimulated electrically.

These methods have the same effect on inhibiting the production of pro-inflammatory cytokines as the previously described methods of activating brain muscarinic receptors, i.e., would inhibit the same pro-inflammatory cytokines, would reduce inflammation in patients with the same inflammatory conditions, and would inhibit the release of pro-inflammatory cytokines from the brain or any peripheral organ or cell served by vagus nerve pathways, for example, the liver or cardiac myocytes.

As previously discussed, activation of brain muscarinic receptors inhibit the release of pro-inflammatory cytokines. By inhibiting the release of pro-inflammatory cytokines, inflammation can be reduced in diseases that are characterized by inflammation mediated by a pro-inflammatory cytokine cascade.

Accordingly, the present invention is directed to methods of treating an inflammatory disease in a vertebrate. The methods comprise activating a brain muscarinic receptor in the vertebrate. The methods are useful for treating any disease in any vertebrate, including humans, that is at least partially mediated by a pro-inflammatory cytokine cascade, including systemic inflammatory diseases. Examples of such diseases have been previously provided. Even though the signal that inhibits the release of pro-inflammatory cytokines is apparently carried by the vagus nerve, these methods are effective in inhibiting systemic inflammatory diseases because the vagus nerve innervates the liver, which is a primary source of pro-inflammatory cytokines in systemic disease.

As previously discussed, the same effect as achieved by activating a muscarinic receptor is also achieved by directly stimulating a vagus nerve pathway in the brain. Thus, the invention is also directed to methods of treating an inflammatory disease in a vertebrate, the methods comprising directly stimulating a vagus nerve pathway in the brain of the vertebrate. As previously discussed, the vagus nerve pathway can be stimulated by any means known in the art, and is useful for treating any inflammatory disease in any vertebrate (including humans) that is at least partially mediated by an inflammatory cytokine cascade.

Since the vagus nerve serves the heart, and since cytokine release is at least partially responsible for myocyte apoptosis in several inflammatory diseases, it is also contemplated that apoptosis of cardiac myocytes can be inhibited in vertebrates, including humans, at risk for cardiac myocyte apoptosis by methods comprising activating a brain muscarinic receptor in the vertebrate. Preferred muscarinic receptors are M1, M2, and M4 receptors. Inflammatory diseases that could be treated by these methods include vasculitis, angiitis, endocarditis, pericarditis, myocarditis, myocardial ischemia, periarteritis nodosa, rheumatic fever, congestive heart failure, adult respiratory distress syndrome, fasciitis, or graft-versus-host disease. As with previously described methods, the brain muscarinic receptor can be activated by administering a muscarinic agonist to the vertebrate, either directly to the brain of the vertebrate, enterically or parenterally. Preferred muscarinic agonists are muscarine, McN-A-343 and MT-3.

Similarly, apoptosis in cardiac myocytes can be inhibited by directly stimulating a vagus nerve pathway in the brain of the vertebrate, for example, electrically.

It has also been discovered that vertebrates can be conditioned to inhibit the release of a pro-inflammatory cytokine by associating the activation of brain muscarinic receptors with a sensory stimulus. Thus, in some embodiments, the invention is directed to methods of conditioning a vertebrate to inhibit the release of a pro-inflammatory cytokine upon experiencing a sensory stimulus. These methods comprise the following steps:

(a) activating a brain muscarinic receptor in the vertebrate and providing the sensory stimulus to the vertebrate within a time period sufficient to create an association between the stimulus and the activation of the brain muscarinic receptor; and

(b) repeating step (a) at sufficient time intervals and duration to reinforce the association sufficiently for the pro-inflammatory cytokine release to be inhibited by the sensory stimulus alone.

These methods are particularly useful for treating chronic inflammatory conditions, such as arthritic conditions, where the methods allow a patient to reduce the need for anti-inflammatory medication. Thus, potential side effects of anti-inflammatory medication, such as gastrointestinal, kidney, heart, or liver effects, can be reduced.

These methods can be used to reduce the release of any of the pro-inflammatory cytokines as with the methods previously discussed, including tumor necrosis factor (TNF), interleukin (IL)-1β, IL-6, IL-18, HMG-B1, MIP-1α, MIP-1β, MIF, interferon-γ, and PAF. In particular, pro-inflammatory cytokine release is inhibited in any organ, tissue, or cell subject to influence by vagus nerve stimulation, including the liver and cardiac myocytes. They are useful for any vertebrate having a vagus nerve, including all mammals. They are particularly useful for vertebrates (including humans) suffering from, or at risk for, a condition mediated by an inflammatory cytokine cascade. Examples of such conditions have been previously discussed.

In the conditioning step of these methods (step (a)), the brain muscarinic receptor can be activated by any means previously discussed. It is believed that the association between the stimulus and the brain muscarinic receptor activation is most effectively created if the stimulus and activation is as close together temporally as possible, preferably within one minute. The time interval between repetitions of the stimulus-activation procedures should also be short enough to optimize the reinforcement of the association. A preferred time interval is twice daily. The duration of the conditioning should also be sufficient to provide optimum reinforcement of the association. A preferred duration is at least one week. Optimum time intervals and durations can be determined by the skilled artisan without undue experimentation by standard methods known in the art.

The sensory stimulus can be from any of the five senses. Nonlimiting examples of suitable sensory stimuli are sounds such as a bell ring, a buzzer, and a musical passage; a touch such as a pin stick, a feather touch, and an electric shock; a taste, or the ingestion of a particular chemical, such as a sweet taste, a sour taste, a salty taste, and saccharine ingestion; a visual image such as a still picture, a playing card, or a short video presentation.

As with previously described methods, the conditioning to inhibit pro-inflammatory cytokine release with a sensory stimulus can utilize stimulation of a vagus nerve pathway in the vertebrate brain rather than activation of brain muscarinic receptors.

Additionally, since inhibiting pro-inflammatory cytokine release also effects a reduction in inflammation, as discussed above, the conditioning methods described above are useful for reducing inflammation in the treated vertebrate. Thus, the present invention is directed to methods of conditioning a vertebrate to reduce inflammation in the vertebrate upon experiencing a sensory stimulus. The methods comprise the following steps:

(a) activating a brain muscarinic receptor in the vertebrate, or directly stimulating a vagus nerve pathway in the brain, and providing the sensory stimulus to the vertebrate within a time period sufficient to create an association between the stimulus and the activation of the brain muscarinic receptor; and

(b) repeating step (a) at sufficient time intervals and duration to reinforce the association sufficiently for the inflammation to be reduced by the sensory stimulus alone.

Preferred embodiments of the invention are described in the following examples.

### EXAMPLE 1

This example describes experiments establishing that CNI-1493 binds to brain muscarinic receptors, that intracerebroventricular (i.c.v.) injections of CNI suppresses carrageenan-induced hindpaw edema and release of TNF into the blood, that these effects are reversed by atropine, and that neither nicotine nor prozak i.c.v. injections inhibits TNF production.

### Methods

Method of determining CNI-1493 receptor binding. CNI-1493 was tested at a single concentration (10 μM) in a panel of receptor binding assays by NovaScreen Biosciences Corporation (Hanover, Md.). Values were expressed as the percent inhibition of specific binding, and represented the average of duplicate tubes.
Method of stereotactic intracerebroventricular injections. A rat model of intracerebroventricular (i.c.v.) injections was established in order to be able to directly deliver pharmacological agents into the brain of rats. This was necessary in order to separate drug effects on peripheral inflammation that occurred through central versus peripheral mechanisms. Lewis rats were anaesthetized with urethane (1 g/kg, i.p.) and xylazine (15 mg/rat, i.m. (intramuscular)). Rats were then placed in a stereotactic head frame (Stoelting, Wood Dale, Ill., USA). The incisor bar was adjusted until the plane defined by the lambda and bregma was parallel to the base plate. For i.c.v. injections the needle of a Hamilton syringe (25 μl) was positioned stereotactically above the lateral ventricle (0.2 mm and 1.5 mm posterior to bregma, 3.2 mm below the dura.) Solutions of the drugs tested were prepared in sterile endotoxin-free water, at the specified concentrations, and a 10-μl injection/rat was administered over 2 min, 1 h prior to either carrageenan injection, or to LPS.

The tested drugs, in either the carrageenan and/or LPS experiments, were: saline control; fluoxetine hydrochloride, (also known as Prozak) (0.01 mg/100 g); muscarine (50 μg/rat, 5 μg/rat, 0.5 μg/rat, 0.05 μg/rat, 0.005 μg/rat); 4-(N-[3-chlorophenyl]carbamoyloxy)-2-butynyltrimethylammonium chloride (also known as McN-A-343) (5 μg/rat); Muscarinic Toxin-3, (also known as MT-3) from Dendroaspis angusticeps snake venom (0.37 μg/rat); nicotine (10 μg/rat); CNI-1493 (1 μg/kg, 50 μg/rat); atropine (1 μg/kg, 5 μg/rat); CNI-1493 plus atropine (1 μg/kg of each of the drugs; 50 μg/rat, 5 μg/rat respectively); naloxone hydrochloride (2 μg/rat), CNI-1493 plus naloxone (50 μg/rat+5 μg/rat respectively); and morphine (20 μg/rat).

Method of carrageenan-induced hindpaw edema. Paw edema was induced in anaesthetized rats by injection of 1% solution of 1-carrageenan (100 μl) into the plantar surface of the left hindpaw. The right hindpaw was injected with the same volume of saline alone (as control). The thickness of the carrageenan-treated and saline-treated hindpaw was measured using a caliper at 3 h post carrageenan, and the difference between paw thickness calculated as an index of inflammation (paw swelling).
Method of LPS injections and TNF determination. LPS (15 mg/kg, i.v.) was injected in the tail vein 1 h after drug injection. Blood was obtained 2 h post LPS injection by paraorbital bleeding. Serum TNF concentrations were determined by an L929 bioactivity assay.
Method of assessing TNF by the L929 bioactivity assay. L929 cells were suspended in Dulbecco's minimal Eagle's medium (DMEM; GibcoBRL) supplemented with fetal bovine serum (10%; Hyclone) and penicillin/streptomycin (0.5%; Sigma Chemical Co.), and plated at 2×10⁴ cells per well in 96-well flat-bottomed microtiter plates. After 24 h, media were respirated and replaced with medium containing cycloheximide (10 μg/ml; Sigma Chemical Co.) and the samples to be assayed/TNF standards. Plates were incubated overnight, at which time cell viability as a function of TNF concentration was assessed by the MTT assay. Absorbance values were converted to units per milliliter by comparison with a standard curve for rat TNF.

### Results

When tested with an in vitro panel of receptor binding assays, CNI-1493 at 10 μM inhibited receptor binding by greater than 50% for seven different receptors, respectively alpha 1 adrenergic (89.7%), muscarinic (60.6%), serotonin (75.6%), Type N calcium channel (84.2%), voltage-insensitive potassium channel (60.2%), voltage-sensitive potassium channel (73.0%), and vasoactive intestinal peptide (58.5%).

CNI-1493 at 10 μM inhibited receptor binding by less than 50% (considered by NovaScreen to be indicative of marginal or no activity) at the following receptors: beta adrenergic, dopamine, glutamate (NMDA agonist site), H1 histamine, Type L calcium channel, chloride channel, site 1 sodium, site 2 sodium, NK1 neurokinin, vasopressin 1, leukotriene D4 and LTD4, thromboxane A2, and epidermal growth factor.

The above-described studies provided a list of receptors to be tested for determination as to whether their alternative pharmacological activation by other drugs would separately cause peripheral immunosuppressive activity, and whether this activity would be further dependent on the efferent vagus nerve. To achieve this purpose, we established an animal model of paw edema and an animal model of endotoxic shock, where the effects of the various drugs were tested by their stereotactic intracerebroventricular delivery into the brain.

In one set of experiments, rats were injected by i.c.v. means with either saline (n=1), CNI-1493 (5 μg/rat, n=3), CNI-1493 plus atropine (5 μg/rat each), or atropine (5 μg/rat). LPS (15 mg/kg, i.v.) was given 1 h later. Blood was collected 2 h post LPS administration. Serum TNF was determined by the L929 assay.

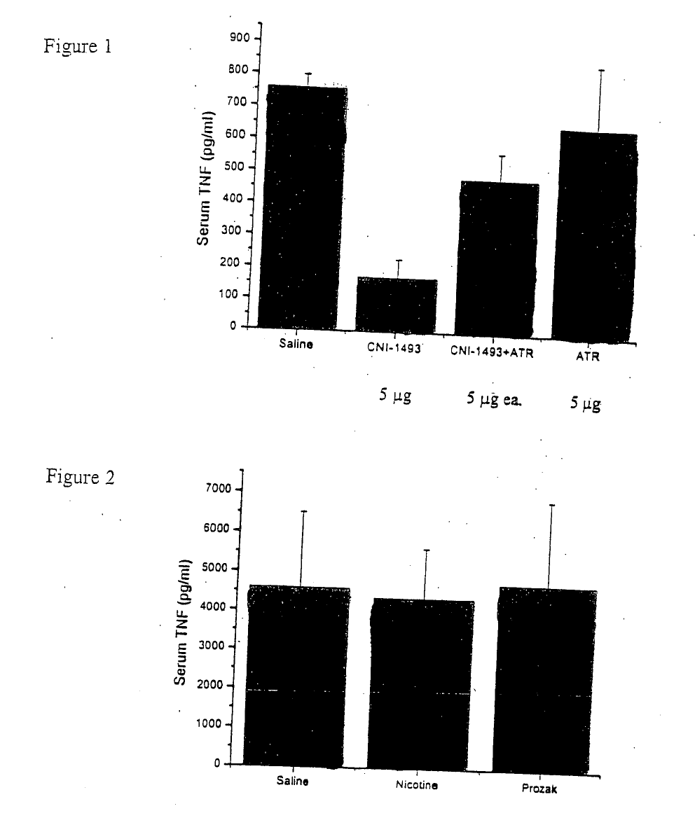

The results of these experiments are summarized in FIG. 1. Intracerebroventicularly administered CNI-1493 inhibited LPS-induced serum TNF levels by more than 80%. Atropine reversed the inhibitory effect of CNI-1493 to the TNF level of atropine alone.

These results indicate that i.c.v. CNI-1493 can suppress peripheral inflammation, and that this effect is reversed by co-administration of i.c.v. atropine. Since atropine is an antagonist at muscarinic receptors, these results thus indicate that the immunosuppressive effects of CNI-1493 are mediated via muscarinic receptors in the brain.

In a second set of experiments, rats were injected by i.c.v. means with either saline (n=4), nicotine (10 μg/rat, n=3), or prozak (0.01 mg/10 g, n=3). LPS (15 mg/kg, i.v.) was given 1 h later. Blood was collected 2 h post LPS administration. Serum TNF was determined by the L929 assay.

The results are summarized in FIG. 2. Neither nicotine nor prozak had any effect in reducing LPS-induced serum TNF levels. These results indicate that neither nicotine nor prozak show central effects on peripheral immunosuppression.

In a third set of experiments, rats were injected by i.c.v. means with either saline (n=4), CNI-1493 (5 μg/rat, n=3), CNI-1493 plus atropine (5 μg/rat each), or atropine (5 μg/rat). Carrageenan was given to the animals 1 h later, and paw edema was determined 3 h post carrageenan.

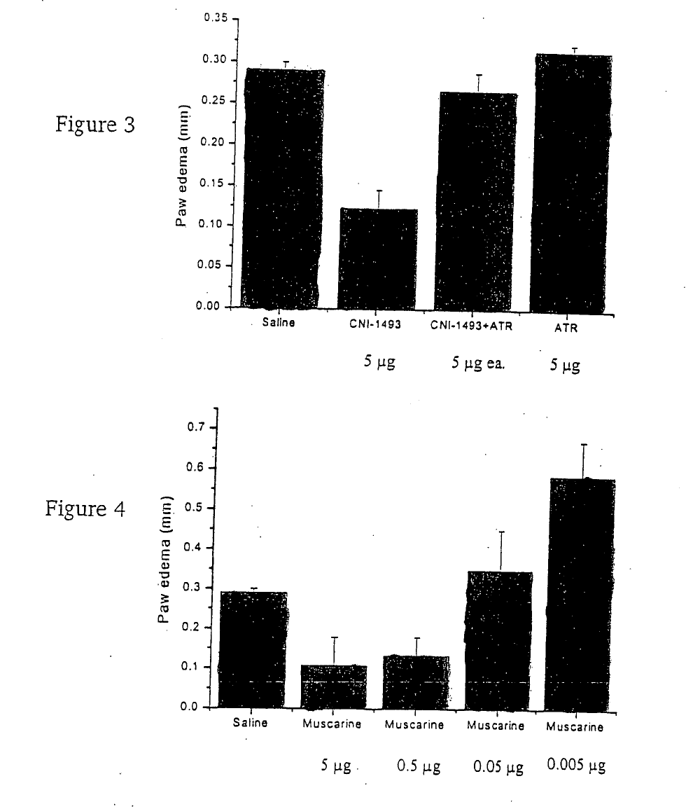

The results of these experiments are summarized in FIG. 3. As with LPS induced serum TNF levels, intracerebroventricular administration of CNI-1493 significantly inhibits carageenan-induced paw edema, and atropine (ATR) reverses the effect.

These results indicate again, by a different method, that i.c.v. CNI-1493 suppresses peripheral inflammation, and that this effect is reversed by co-administration of i.c.v. atropine. Since atropine is an antagonist at muscarinic receptors, these results thus indicate that the immunosuppressive effects of CNI-1493 are mediated via muscarinic receptors in the brain.

In another set of experiments, rats were injected by i.c.v. means with either saline, or muscarine (from left to right on the bar graph-5 μg/rat, 0.5 μg/rat, 0.05 μg/rat, 0.005 μg/rat, n=4 animals/group). Carrageenan was given to the animals 1 h later, and paw edema was determined 3 h post carrageenan.

FIG. 4 summarizes the results of these experiments. Intracerebroventricular administration of muscarine significantly inhibits carrageenan-induced paw edema in a dose-dependent manner. These results further establish that i.c.v. muscarine produces peripheral suppression of inflammation.

In other experiments, rats were subjected to bilateral cervical vagotomy (VGX) or alternatively to bilateral vagus nerve isolation. Intracerebroventricular injections were then performed (26-66 min. later) in each of the four groups of either saline (SAL, n=2 animals/group), or muscarine (MUS, 0.5 μg/rat, n=4 animals/group). Carrageenan was given to the animals 1 h post the i.c.v. drug injections, and paw edema was determined 3 h post carrageenan. P=0.015 SAL v. MUS. P=0.039 MUS v. MUS-VGX.

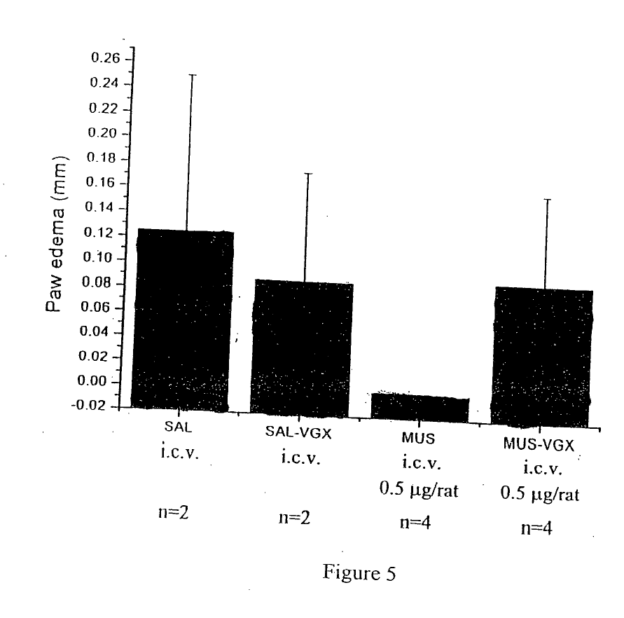

FIG. 5 summarizes the results of these experiments. Vagotomy clearly abrogates the inhibitory effects of intracerebroventricular (i.c.v.) administration of muscarine on carrageenan-induced paw edema. Thus, vagotomy abrogates the peripheral immunosuppressive effects of centrally administered muscarine, establishing that activation of muscarinic receptors in the brain carries a peripheral immunosuppressive signal through the vagus nerve.

### EXAMPLE 2

This example provides experimental results establishing the preferred muscarinic receptor subtypes useful for the present invention.

### Methods

Method of determining muscarinic receptor subtype. CNI-1493 was tested at a single concentration (10 μM) in a panel of muscarinic receptor binding assays by NovaScreen Biosciences Corporation (Hanover, Md.). Values were expressed as the percent inhibition of specific binding, and represented the average of duplicate tubes.

Other methods are as described in Example 1.

### Results

Table 1 summarizes the results of testing of CNI-1493 for inhibiting binding to a panel of muscarinic receptors as indicated.

TABLE 1

| Receptor | Percent inhibition |
|---|---|
| Muscarinic, M1 | 83% |
| Muscarinic, M1 (Human recombinant) | 72% |
| Muscarinic, M2 | 85% |
| Muscarinic, M2 (Human recombinant) | 58% |
| Muscarinic, M3 | 9% |
| Muscarinic, M3 (Human recombinant) | 40% |
| Muscarinic, M4 (Human recombinant) | 57% |
| Muscarinic, M5 (Human recombinant) | 43% |

Values of less than 50% are considered by NovaScreen to show marginal or no activity.

This results indicate that M1, M2, and M4 are the primary muscarinic receptors that bind to CNI-1493.

In another set of experiments, animals were injected by i.c.v. as described in Example 1 with either saline, the M1 agonist McN-A-343 (5 μg/rat, n=5), or the M4-agonist MT-3 (0.37 μg/rat, n=4). Carrageenan was given to the animals 1 h later as described in Example 1, and paw edema was determined 3 h post carrageenan administration.

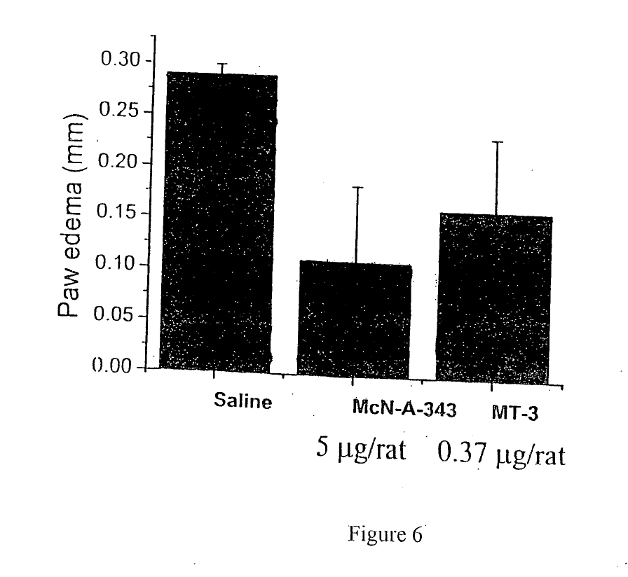

The results of these experiments are provided in FIG. 6. Intracerebroventricular administration of the M1 agonist McN-A-343 or the M4 agonist MT-3 significantly inhibits carrageenan-induced paw edema. These results further establish that central activation of M1 and M4 receptors plays a role in suppressing peripheral immune processes.

In other experiments, animals were injected i.c.v. with either saline, or the M1 agonist McN-A-343 at 5 μg/rat (n=5). Alternatively, McN-A-343 was given peripherally at a much higher concentration (5 mg/kg, i.p., n=2). Carrageenan was given to the animals 1 h post i.c.v. or i.p. drug administration, and paw edema was determined 3 h post carrageenan.

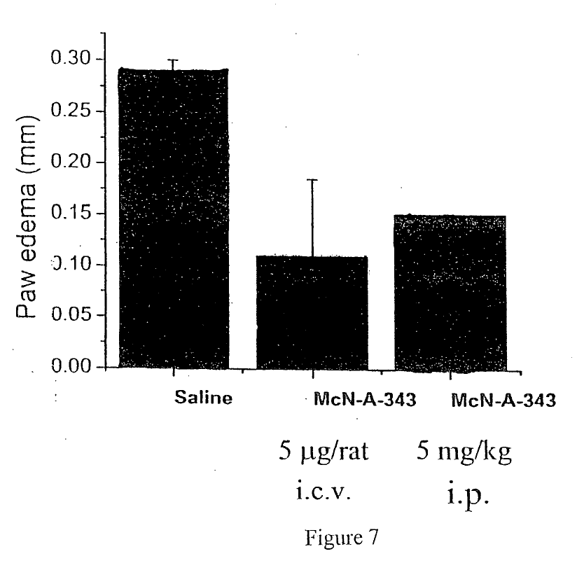

Results of these experiments are summarized in FIG. 7. Intracerebroventricular (i.c.v.) administration of the M1 agonist McN-A-343 has a comparable effect on inhibition of carrageenan-induced paw edema as a higher dose administered intraperitoneally (i.p.). These results indicate that the significantly higher i.p. concentration of an M1 agonist that is needed to achieve peripheral immunosuppression is attributable to a small degree of blood brain barrier penetration of this compound. Thus, it is likely that the small amount of centrally penetrated compound that is responsible for the observed immunosuppressive effects of the drug.

### EXAMPLE 3

This Example provides experimental results that indicate that mammals can be conditioned to mount an anti-inflammatory response through a sensory stimulus that has been associated with activation of brain muscarinic receptors.

### Methods

Mice were grouped into four groups (n=4 animals/group). The conditioning training for Groups 2-4 consisted of morning and afternoon sessions. Mice in group 2 were together taken to a room, where each mouse was injected with CNI-1493 (2.5 mg/kg, i.p.). Simultaneously with the injection, each mouse was subjected to 45 seconds of bell ringing. Group 4 mice, similar to Group 2 mice, were subjected to control conditioning, whereby mice were injected with saline, instead of CNI-1493. Group 3 mice, like Group 2 mice, were subjected to saline injections but not bell ringing. This protocol was performed over a 10 day period, on days 1-4 and 8-10. On day 11, Group 1 mice were injected with CNI-1493 (2.5 mg/kg, i.p.). Also on day 11, 30 min after the Group 1 mice injections were performed, animals in all groups were injected with LPS (5 mg/kg, i.p.). After 2 hours, the mice were euthanized via CO₂ inhalation, and blood was withdrawn. Serum TNF was determined by the L929 assay.

### Results

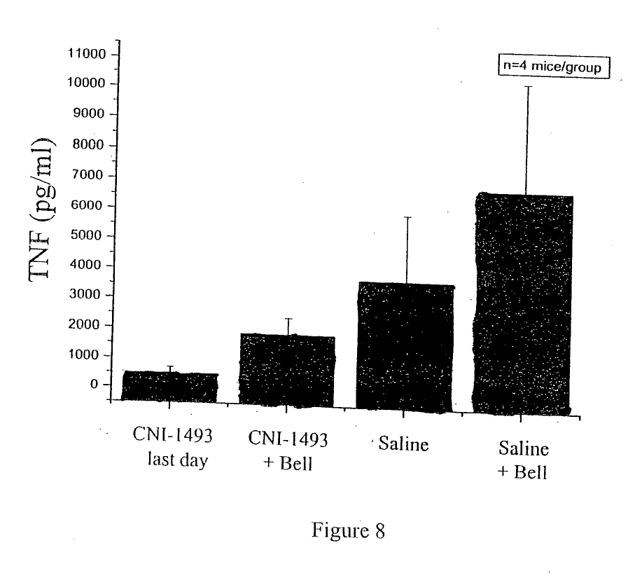

The results of this experiment are summarized in FIG. 8. The mean LPS-induced TNF release was reduced by about 60% in animals conditioned by associating repeated intraperitoneal CNI-1493 administration with bell ringing vs. animals exposed to bell ringing and intraperitoneal saline injections (Group 2 vs. Group 4; p=0.22)

On the basis of these experiments, immunosuppression mediated via stimulation of the efferent vagus nerve can be expected to be achieved by conditioned exposure to a neutral stimulus (i.e., bell) following conditioning training with a neutral stimulus and a drug known to activate brain muscarinic receptors (here, CNI-1493).

### EXAMPLE 4

This Example provides experimental results that indicate that intracerebroventricular administration of muscarine into rats causes a dose-dependent decrease in serum, spleen, and heart TNF concentrations.

### Methods

Methods of stereotactic intracerebroventricular injection of muscarine into rats and LPS injections were as described in Example 1. TNF levels in serum and tissues were determined using an enzyme-inked immunosorbent assay (ELISA) according to the manufacturere's instructions (R & D Systems (Minneapolis, Minn.)).

### Results

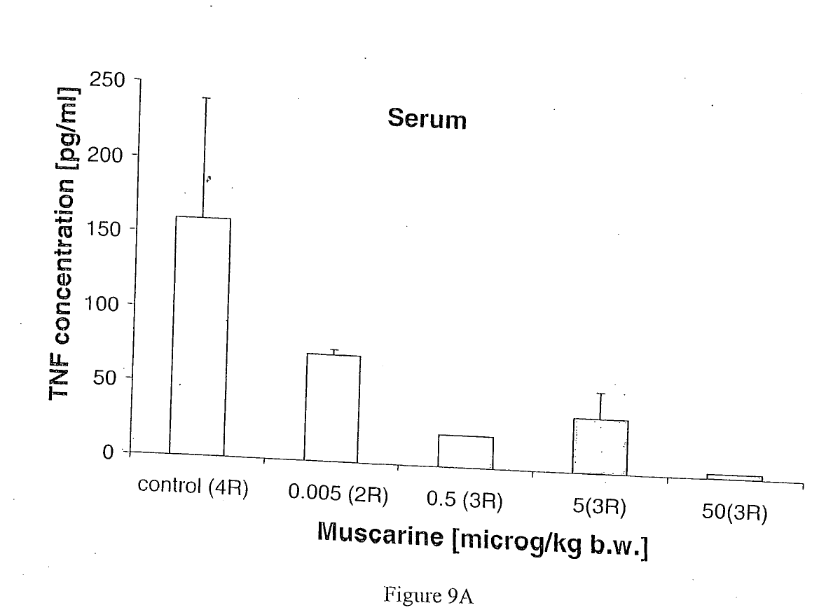

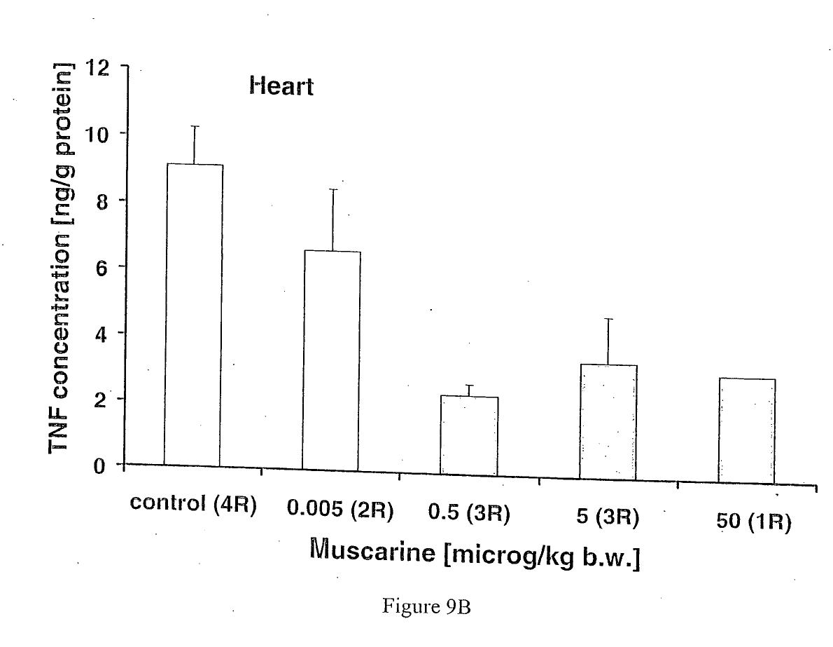

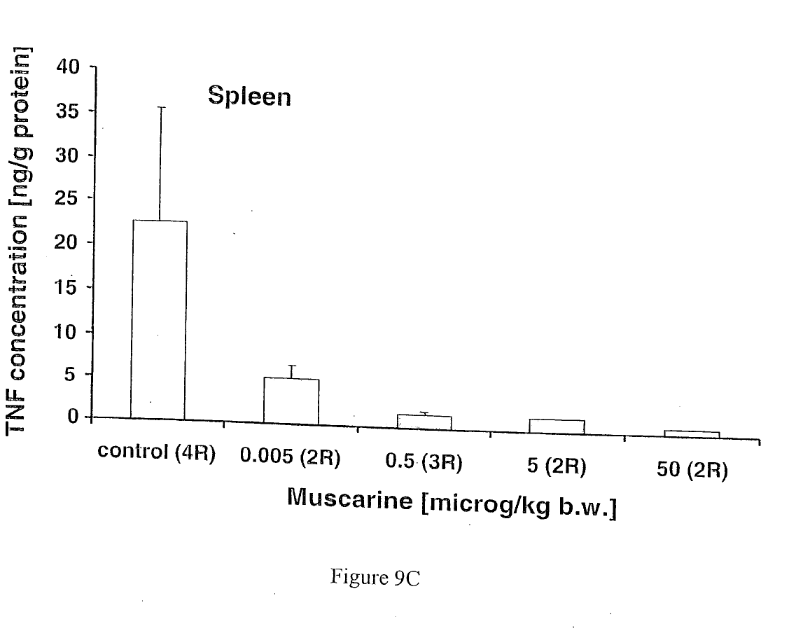

Rats were injected by i.c.v. means with either saline (control) or muscarine (0.005 μg/kg body weight, 0.5 μg/kg body weight, 5.0 μg/kg body weight, or 50 μg/kg body weight). LPS was administered 1 hour later. Two hours after LPS administration the rats were sacrificed and blood, heart tissue, and spleen tissue were isolated from the rats. The results of these experiments are summarized in FIGS. 9A-9C. As shown in FIGS. 9A-9C, i.c.v. administration of muscarine inhibited LPS-induced serum, heart, and spleen (peripheral) TNF levels. These results demonstrate that peripheral TNF production can be inhibited by the activation of central muscarinic receptors.

### EXAMPLE 5

This Example provides experimental results that indicate that intravenous administration of muscarine into rats has no effect on rat spleen, liver, and heart TNF concentrations.

### Methods

Methods of LPS injections were as described in Example 1. Determination of serum and tissue TNF levels were as described in Example 4. Muscarine (or control saline) was intravenously injected into rats at concentrations of 0.05 μg/kg body weight, 0.5 μg/kg body weight, or 5.0 μg/kg body weight.

### Results

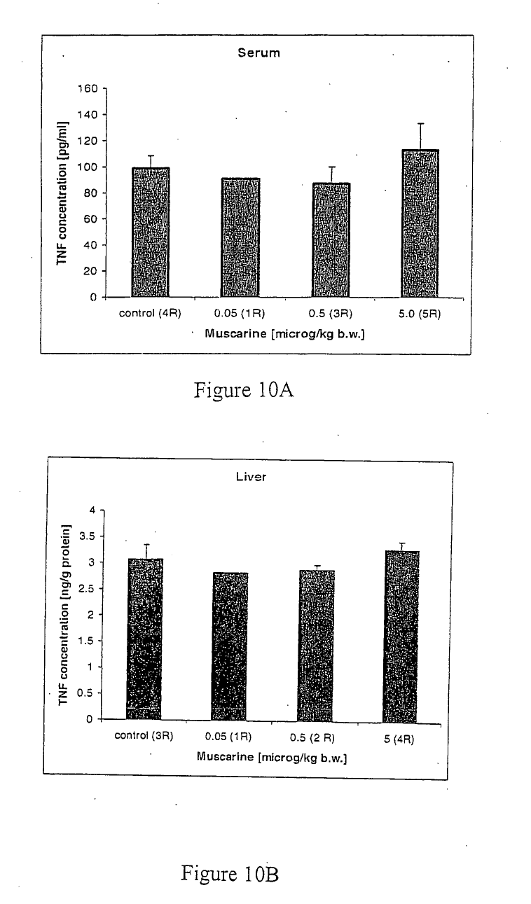

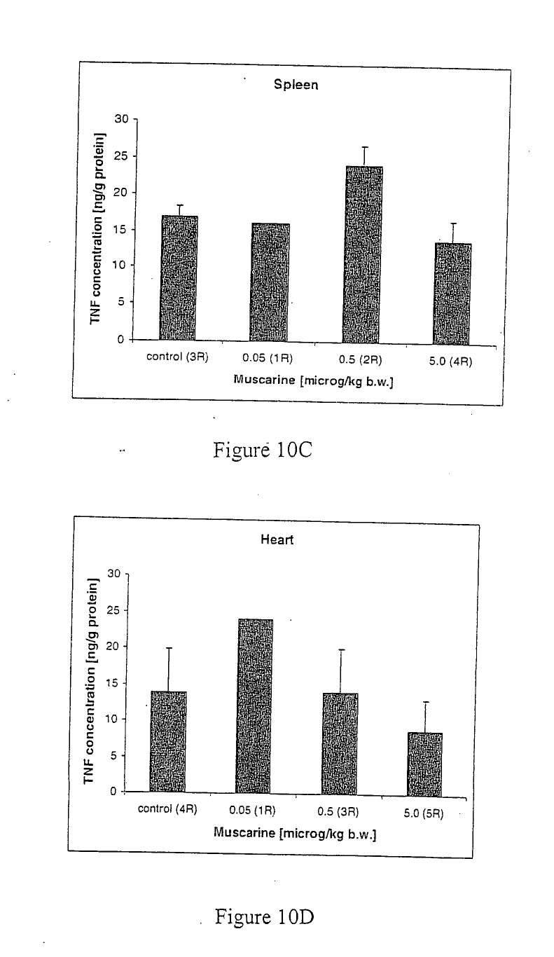

Rats were injected by i.v. means with either saline (control) or muscarine (0.05 μg/kg body weight, 0.5 μg/kg body weight, or 5.0 μg/kg body weight). LPS was administered 1 hour later. Two hours after LPS administration the rats were sacrificed and blood, liver tissue, heart tissue, and spleen tissue were isolated from the rats and assayed for TNF concentrations. The results of these experiments are summarized in FIGS. 10A-10D. As shown in FIGS. 10A-10D, intravenous administration of muscarine had no effect on LPS-induced serum, liver, heart, and spleen TNF levels.

Muscarine is a quarternary salt, and as such it does not readily cross the blood brain barrier. The above results demonstrate that the activation of peripheral muscarinic receptors has no effect on LPS induced TNF production.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are incorporated herein by reference. The discussion of the references herein is intended merely to summarize the assertions made by the authors and no admission is made that any reference constitutes prior art. Applicants reserve the right to challenge the accuracy and pertinence of the cited references.

While this invention has been particularly shown and described with references to preferred embodiments thereof, it will be understood by those skilled in the art that various changes in form and details may be made therein without departing from the scope of the invention encompassed by the appended claims.

### REFERENCES CITED

Antonica, A., et al., J. Auton. Nerv. Syst., 48:187-97, 1994.
Bhattacharya S. K., et al., Res. Exp. Med. 191:65-76, 1991.
Besedovsky, H., et al., Science, 233:652-54, 1986.
Blackwell, T. S., and Christman, J. W., Br. J. Anaesth., 77: 110-17, 1996.
Blum, A. and Miller, H., Am. Heart J, 135:181-86, 1998.
Borovikova, L. V., et al., Nature, 405: 458-62, 2000a.
Borovikova, L. V., et al., Auton. Neurosci., 20:141-47, 2000b.
Bumgardner, G. L., and Orosz, C. G., Semin. Liver Dis., 19: 189-204, 1999.
Carteron, N. L., Mol. Med. Today, 6:315-23, 2000.
Dibbs, Z., et al., Proc. Assoc. Am. Physicians, 111:423-28, 1999.
Dinarello, C. A., FASEB J, 8:1314-25, 1994.
Fleshner, M., et al., J. Neuroimmunol., 86:134-41, 1998.
Fox, D. A., Arch. Intern. Med., 28:437-444, 2000.
Gattorno, M., et al., J. Rheumatol., 27:2251-2255, 2000.
Gaykema, R. P., et al., Endocrinology, 136:4717-4720, 1995.
Gracie, J. A., et al., J. Clin. Invest., 104:1393-1401, 1999.
Green, E., Psycophysiology, 6, 371-377, 1969.
Gregory, S. H. and Wing, E. J., Immunology Today, 19:507-10, 1998.
Guslandi, M., Br. J. Clin. Pharmacol., 48:481-84, 1999.
Hirano, T., J. Surg. Res., 81:224-29, 1999.
Hommes, D. W. and van Deventer, S. J., Curr. Opin. Clin. Nutr. Metab. Care, 3:191-95, 2000.
Hsu, H. Y., et al., J. Pediatr. Gastroenterol., 29:540-45, 1999.
Hu, X. X., et al., J. Neuroimmunol., 31:35-42, 1991.
Jander, S, and Stoll, G., J. Neuroimmunol., 114:253-58, 2001.
Kanai, T. et al., Digestion, 63 Suppl. 1:37-42, 2001.
Katagiri, M., et al., J. Clin, Gastroenterol., 25 Suppl. 1: S211-14, 1997.
Kimmings, A. N., et al., Eur. J. Surg., 166:700-05, 2000.
Kumins, N. H., et al., SHOCK, 5:385-88, 1996.
Lee, H. G., et al., Clin. Exp. Immunol., 100:139-44, 1995.
Lipton, J. M. and Catania, A., Immunol. Today, 18:140-45, 1997.
Madretsma, G. S., et al., Immunopharmacology, 35:47-51, 1996.
McGuinness, P. H., et al., Gut, 46:260-69, 2000.
Nathan, C. F., J. Clin. Invest., 79:319-26, 1987.
Pulkki, K. J., Ann. Med., 29:339-43, 1997.
Prystowsky, J. B. and Rege, R. V., J. Surg. Res., 71; 123-26 1997.
Rayner, S. A. et al., Clin. Exp. Immunol., 122:109-16, 2000.
Romanovsky, A. A., et al., Am. J. Physiol., 273:R407-13, 1997.
Sandborn, W. J., et al., Ann. Intern. Med, 126:364-71, 1997.
Sato, E., et al., Am. J. Physiol., 274:L970-79, 1998.
Sato, K. Z., et al., Neurosci. Lett., 266:17-20, 1999.
Scheinman, R. I., et al., Science, 270:283-86, 1995.
Sher, M. E., et al., Inflamm. Bowel Dis., 5:73-78, 1999.
Sternberg, E. M., J. Clin. Invest., 100:2641-47, 1997.
Thompson, A., Ed. The Cytokine Handbook, 3^{rd} ed., Academic Press, 1998.
Tracey, K. J. et al., Nature, 330:662-64, 1987.
Tracey, K. J. et al., Science, 234:470-74, 1986.
Tsutsui, H., et al., Immunol. Rev., 174:192-209, 2000.
van Dijk, A. P., et al., Eur. J. Clin. Invest., 28:664-71, 1998.
Wang, H., et al., Science, 285:248-51, 1999.
Waserman, S., et al., Can. Respir. J, 7:229-37, 2000.
Watanabe, H. et al., J. Reconstr. Microsurg., 13:193-97, 1997.
Wathey, J. C., et al., Biophys. J, 27:145-64, 1979.
Watkins, L. R. and Maier, S. F., Proc. Natl. Acad. Sci. U.S.A., 96:7710-13, 1999.
Watkins L. R., et al., Neurosci. Lett. 183:27-31, 1995.
Whaley, K., et al., Nature, 293:580-83, 1981.
Woiciechowsky, C., et al., Nat. Med., 4: 808-13, 1998.
Yeh, S. S., and Schuster, M. W., Am. J. Clin. Nutr., 70, 183-97, 1999.
Zhang and Tracey, in The Cytokine Handbook, 3^{rd} ed., Ed. Thompson, Academic Press, 515-47, 1998.
PCT patent publication WO 00/47104.

What is claimed is:

1. A method of inhibiting release of a pro-inflammatory cytokine in a vertebrate at risk for or having a condition mediated by an inflammatory cytokine cascade, the method comprising directly stimulating a vagus nerve pathway in the brain of the vertebrate.

2. The method of claim 2, wherein the vagus nerve pathway is stimulated electrically.

3. A method of treating an inflammatory disease in a vertebrate, the method comprising directly stimulating a vagus nerve pathway in the brain of the vertebrate in an amount sufficient to inhibit release of a pro-inflammatory cytokine in the vertebrate.

4. The method of claim 3, wherein the vagus nerve pathway is stimulated electrically.
